(19) United States
(12) Patent Application Publication
(10) Pub. No.: US 2008/0227992 A1
(43) Pub. Date: Sep. 18, 2008

(54) Catalyst and Methods for Producing Maleic Anhydride

(75) Inventors: Cornelia Dobner, Ludwigshafen (DE); Mark Duda, Ludwigshafen (DE); Andreas Raichle, Ludwigshafen (DE); Hagen Wilmer, Ludwigshafen (DE); Frank Rosowski, Mannheim (DE); Markus Holzle, Kirchheim (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 11/997,023

(22) PCT Filed: Jul. 21, 2006
(86) PCT No.: PCT/EP2006/064551

§ 371 Date: Jan. 28, 2008

(30) Foreign Application Priority Data
Jul. 28, 2005 (DE) 10 2005 035 978.7

(51) Int. Cl. C07D 307/60; B01J 27/198; B01J 27/16

(52) U.S. Cl. 549/260; 502/209

(57) ABSTRACT

Catalysts comprising a catalytically active composition, the catalytically active composition comprising vanadium, phosphorus, iron and oxygen, wherein the catalytically active composition has an iron:vanadium atomic ratio of 0.005 to <0.05, and wherein iron in the catalytically active composition is derived from an iron starting material comprising Fe(III) phosphate; and processes for making such catalysts as well as uses therefor to prepare maleic anhydride are described.

The present invention relates to a catalyst for preparing maleic anhydride by heterogeneously catalyzed gas-phase oxidation of a hydrocarbon having at least four carbon atoms, which comprises a catalytically active composition comprising vanadium, phosphorus, iron and oxygen, wherein the catalytically active composition has an iron/vanadium atomic ratio of from 0.005 to <0.05 and Fe(III) phosphate is used as iron starting material. The invention further relates to a number of processes for producing the catalyst of the invention and the use of the catalyst in the preparation of maleic anhydride.

Maleic anhydride is an important intermediate in the synthesis of γ-butyrolactone, tetrahydrofuran and 1,4-butanediol, which are in turn used as solvents or are, for example, processed further to form polymers such polytetrahydrofuran or polyvinylpyrrolidone.

In the prior art, the use of doped metals such as molybdenum, zinc, hafnium, zirconium, titanium, chromium, nickel, copper, boron, silicon, antimony, niobium, bismuth, iron, copper, manganese, aluminum, lithium, cerium, bismuth, tin, gallium or cobalt in vanadyl phosphate (VPO) catalysts is reported in general terms.

Adelouahab et al. (Chem. Soc., Faraday Trans. (1995), 91, 3231-3235) disclose VPO catalysts doped with iron and cobalt for preparing maleic anhydride. An Fe/V ratio of from 0.02 to 0.05 is examined. The BET surface area of the doped catalysts is not more than 14 m²/g. As iron component, use is made of iron acetylacetonate. It is stated that the iron acetylacetonate and cobalt acetylacetonate is dissolved in isobutanol before refluxing of the vanadium compound.

Sananes-Schultz et al. (J. Catal. (1996) 163, 346-353) describe VPO catalysts doped with iron and cobalt. A catalyst having an Fe/V ratio of 0.05 is examined. As iron component, use is made of iron acetylacetonate. It is stated that the undoped catalyst precursor is prepared by refluxing in an aqueous solution. The catalyst precursor is filtered and then washed and refluxed again in isobutanol in which iron and cobalt have been dissolved. It is stated that the doping of cobalt has a positive effect. In contrast, no effect was found in the case of iron doping.

Mastuura et al. (Stud. Surf. Catal. (1995), 92, 185-190) disclose a VPO catalyst doped with iron(II) or iron(II). A catalyst having an Fe/V ratio of from 0.05 to 0.4 is described. As iron component, use is made of iron phosphate. The doped catalyst is produced by mixing the catalyst precursor VO(HPO₄)*0.5H₂O and Fe₃(PO₄)₂*nH₂O and stirring the mixture in toluene. The toluene is evaporated and the catalyst is calcined/activated in the presence of butane. It is stated that better results are achieved when using iron(II) than when using iron(III).

EP-A 92 619, EP-A 458 541 and U.S. Pat. No. 4,244,878 disclose the use of the dopant metal zinc in VPO catalysts. Here, zinc is used in a ratio of zinc/vanadium of from 0.001 to 0.4.

WO 97/12674 and U.S. Pat. No. 5,506,187 describe doping of the VPO catalyst with molybdenum. WO 97/12674 discloses VPO catalysts having a molybdenum/vanadium molar ratio of from 0.0020 to 0.0060, with molybdenum being concentrated essentially on the surface of the catalyst.

U.S. Pat. No. 4,062,873, U.S. Pat. No. 4,699,985 and U.S. Pat. No. 4,442,226 disclose the use of silicon in VPO catalysts; in U.S. Pat. No. 4,699,985 and U.S. Pat. No. 4,442,226 doping is additionally carried out using at least one further dopant metal selected from among indium, tantalum and antimony.

DE-A 30 18 849 describes a VPO catalyst doped with zinc, lithium and silicon.

U.S. Pat. No. 5,364,824 discloses a VPO catalyst which is doped with, for example, bismuth or zirconium in a ratio of dopant metal to vanadium of from 0.007 to 0.02.

WO 00/44494 describes a process for activating VPO catalysts. Catalysts of this type doped with bismuth or with zinc, lithium and molybdenum displayed particularly good results. The dopant metals were used in a ratio of dopant metal to vanadium of from 0.001 to 0.15.

EP-A 458 541, EP-A 655 951 and U.S. Pat. No. 5,446,000 disclose a VPO-Zn—Li catalyst doped with from 0.005 to 0.025 mol or from 0.001 to 0.1 mol of molybdenum per mole of vanadium. U.S. Pat. No. 5,922,637 describes a VPO catalyst doped with zinc, lithium and/or molybdenum.

EP-A 221 876 describes VPO catalysts which further comprise iron and lithium in a ratio of iron/vanadium of from 0.001 to 0.004 and of lithium/vanadium of from 0.0015 to 0.004. The catalyst is produced by reacting a component comprising predominantly tetravalent vanadium with a phosphorus component and a promoter component comprising iron and lithium in a water-free alcohol in the presence of a chloride. It is stated that a maleic anhydride yield of from 48.5 to 54.5% and a maleic anhydride selectivity of from 67.3 to 69.8% are achieved.

U.S. Pat. No. 5,543,532 discloses a VPO catalyst comprising, as further promoters, antimony and also iron, copper, manganese, aluminum, lithium, cerium, bismuth, tin, gallium or cobalt. Use is made predominantly of VPO catalysts doped with antimony and iron, with the iron being present in a ratio of iron/vanadium of from 0.01 to 0.08. Doping is effected together with the reduction of V₂O₅ in water-free alcohols. Selectivities of from 15 to 74% at a conversion of 40% are described.

Despite the comprehensive prior art in the field of VPO catalyst research, there continues to be a need for optimization with a view to an improved yield at comparable activity.

It was accordingly an object of the present invention to provide a catalyst which gives a high yield at an activity comparable to the prior art. A further object was to discover inexpensive doping components. Furthermore, a process for producing doped catalysts which makes it possible to introduce the doping component preferably without use of an excess of this doping component was to be provided. Furthermore, the amount of solvent used in the doping procedure was to be reduced.

We have found a catalyst for preparing maleic anhydride by heterogeneously catalyzed gas-phase oxidation of a hydrocarbon having at least four carbon atoms, which comprises a catalytically active composition comprising vanadium, phosphorus, iron and oxygen, wherein the catalytically active composition has an iron/vanadium atomic ratio of from 0.005 to <0.05 and Fe(III) phosphate is used as iron starting material.

The catalytically active composition advantageously has an iron/vanadium atomic ratio of from 0.01 to 0.035.

The catalysts of the invention advantageously have a phosphorus/vanadium atomic ratio of from 0.9 to 1.5, preferably from 0.9 to 1.2, in particular from 1.0 to 1.1. The average oxidation state of the vanadium is advantageously from +3.9 to +4.4 and preferably from 4.0 to 4.3. The catalysts of the invention advantageously have a BET surface area of from >15 m²/g, preferably from >15 to 50 m²/g and in particular from >15 to 40 m²/g. They advantageously have a pore volume of >0.1 ml/g, preferably from 0.15 to 0.5 ml/g and in particular from 0.15 to 0.4 ml/g. The bulk density of the catalysts of the invention is advantageously from 0.5 to 1.5 kg/l and preferably from 0.5 to 1.0 kg/l.

The catalysts of the invention can comprise the active composition comprising vanadium, phosphorus, iron and oxygen in, for example, pure, undiluted form as “all-active catalyst” or diluted with a preferably oxidic support material as “mixed catalysts”. Suitable support materials for the mixed catalysts are, for example, aluminum oxide, silicon dioxide, aluminosilicates, zirconium dioxide, titanium dioxide or mixtures thereof. Preference is given to all-active catalysts.

An all-active catalyst can have any shape, with preference being given to cylinders, hollow cylinders, trilobes, in particular hollow cylinders as described, for example, in EP-A 1 487 576 and EP-A 552 287.

The external diameter d₁ of the catalyst of the invention is advantageously from 3 to 10 mm, preferably from 4 to 8 mm, in particular from 5 to 7 mm. The height h is advantageously from 1 to 10 mm, preferably from 2 to 6 mm, in particular from 3 to 5 mm. The diameter of the through-opening d₂ is advantageously from 1 to 8 mm, preferably from 2 to 6 mm, very particularly preferably from 2 to 4 mm.

Furthermore, the catalysts of the invention can comprise further promoters; lithium and antimony are advantageously excepted. The elements of groups 1 to 15 of the Period Table and compounds thereof are useful as promoters. Suitable promoters are described, for example, in WO 97/12674 and WO 95/26817 and also in U.S. Pat. No. 5,137,860, U.S. Pat. No. 5,296,436, U.S. Pat. No. 5,158,923 and U.S. Pat. No. 4,795,818, As further promoters, preference is given to compounds of the elements molybdenum, zinc, hafnium, zirconium, titanium, chromium, manganese, nickel, copper, boron, silicon, tin, niobium, cobalt and bismuth, in particular molybdenum, zinc, bismuth. The catalysts of the invention can comprise one or more further promoters. The total content of promoters in the finished catalyst is generally not more than about 5% by weight in each case calculated as oxide.

The catalysts of the invention can also comprise auxiliaries such as tableting aids or pore formers as described, for example, in EP-B 1 261 424 in paragraphs [0021] and [0022].

The present invention further provides a process for producing an iron-doped catalyst for preparing maleic anhydride, where the catalytically active composition has an iron/vanadium atomic ratio of from 0.005 to 0.1, (process 1), which comprises
a) Reacting a pentavalent vanadium compound (e.g. V₂O₅) and iron(III) phosphate with an organic, reducing solvent (e.g. an alcohol such as isobutanol) in the presence of a pentavalent phosphorus compound (e.g. orthophosphoric and/or pyrophosphoric acid) with heating to from 75 to 205° C. If appropriate, this step can be carried out in the presence of a dispersed, pulverulent support material. The reaction is preferably carried out without additional support material.
b) Isolating the vanadium-, phosphorus-, iron- and oxygen-comprising catalyst precursor formed (“VPO precursor”), e.g. by filtration or evaporation.
c) Drying the VPO precursor and/or subjecting it to preliminary heat treatment, if appropriate partial preactivation by additional elimination of water from the VPO precursor. Drying is generally a thermal treatment in the range from 30 to 250° C. which is generally carried out at a pressure of from 0.0 (“vacuum”) to 0.1 MPa abs (“atmospheric pressure”) (cf. WO 03/078059, page 15, lines 12 to 28). Preliminary heat treatment is generally a thermal treatment in the range from 200 to 350° C., preferably from 250 to 350° C. (cf. WO 03/078059, page 15, line 30 to page 16, line 5). Pulverulent support material and/or a pore former, in particular substances which decompose at below 250° C. without leaving a residue, can, if appropriate, then be mixed into the dried VPO precursor powder. Preference is given to carrying out further processing without addition of a support material.
d) Shaping of the VPO precursor to give it a spherical, ring-shaped or shell-like structure for example. Shaping is preferably carried out by tableting, advantageously with prior mixing-in of a lubricant such as graphite (e.g. finely divided graphite from Timcal AG (San Antonio, USA) of the grade TIMREX P 44 (sieve analysis: min. 50% by weight <24 mm, max. 10% by weight >24 μm and <48 μm, max. 5% by weight >48 μm, BET surface area: from 6 to 13 m²/g).
e) Preactivating the shaped VPO precursor by heating in an atmosphere, comprising oxygen, nitrogen, noble gases, carbon dioxide, carbon monoxide and/or water vapor. The mechanical and catalytic properties of the catalyst can be influenced by means of a suitable combination of temperatures, treatment times and gas atmospheres matched to the respective catalyst system.

The present invention further provides a process for producing an iron-doped catalyst for preparing maleic anhydride, where the catalytically active composition has an iron/vanadium atomic ratio of from 0.005 to 0.1, (process 2), which comprises
a₁) reacting a pentavalent vanadium compound (e.g. V₂O₅) with an organic, reducing solvent (e.g. an alcohol such as isobutanol) in the presence of a pentavalent phosphorus compound (e.g. orthophosphoric and/or pyrophosphoric acid) with heating to from 75 to 205° C., preferably to from 100 to 120° C.,
a₂) cooling the reaction mixture to advantageously from 40 to 90° C.,
a₃) adding a divalent or trivalent iron compound (e.g. iron phosphate) and
a₄) heating the reaction mixture to from 75 to 205° C., preferably from 100 to 120° C., again.
b) Isolating the vanadium-, phosphorus-, iron- and oxygen-comprising catalyst precursor formed (“VPO precursor”), e.g. by filtration or evaporation.
c) Drying the VPO precursor and/or subjecting it to preliminary heat treatment, if appropriate partial preactivation by additional elimination of water from the VPO precursor. Drying is generally a thermal treatment in the range from 30 to 250° C. which is generally carried out at a pressure of from 0.0 (“vacuum”) to 0.1 MPa abs (“atmospheric pressure”) (cf. WO 03/078059, page 15, lines 12 to 28). Preliminary heat treatment is generally a thermal treatment in the range from 200 to 350° C., preferably from 250 to 350° C. (cf. WO 03/078059, page 15, line 30 to page 16, line 5). Pulverulent support material and/or a pore former, in particular substances which decompose at below 250° C. without leaving a residue, can, if appropriate, then be mixed into the dried VPO precursor powder. Preference is given to carrying out further processing without addition of a support material.
d) Shaping of the VPO precursor to give it a spherical, ring-shaped or shell-like structure for example. Shaping is preferably carried out by tableting, advantageously with prior mixing-in of a lubricant such as graphite (e.g. finely divided graphite from Timcal AG (San Antonio, USA) of the grade TIMREX P 44 (sieve analysis: min. 50% by weight <24 mm, max. 10% by weight >24 μm and <48 μm, max. 5% by weight >48 μm, BET surface area: from 6 to 13 m²/g).
e) Preactivating the shaped VPO precursor by heating in an atmosphere, comprising oxygen, nitrogen, noble gases, carbon dioxide, carbon monoxide and/or water vapor. The mechanical and catalytic properties of the catalyst can be influenced by means of a suitable combination of temperatures, treatment times and gas atmospheres matched to the respective catalyst system.

The iron is advantageously used in the form of Fe(III) phosphate, Fe(III) acetylacetonate, Fe oxide such as FeO, FeOOH or Fe₂O₃, Fe vanadate, Fe molybdate, Fe(III) citrate or Fe(II) oxalate. It is also possible to use mixtures of iron compounds. The iron is preferably used in the form of iron molybdate or iron phosphate. The iron is particularly preferably used as iron(III) phosphate.

The present invention further provides a process for producing an iron-doped catalyst for preparing maleic anhydride, where the catalytically active composition has an iron/vanadium atomic ratio of from 0.005 to 0.1, (process 3), in which the steps a) and c) comprise the following:
a) Reacting a pentavalent vanadium compound (e.g. V₂O₅) with an organic, reducing solvent, (e.g. an alcohol such as isobutanol) in the presence of a pentavalent phosphorus compound (e.g. orthophosphoric and/or pyrophosphoric acid) with heating.
b) Isolating the vanadium-, phosphorus-, iron- and oxygen-comprising catalyst precursor formed (“VPO precursor”), e.g. by filtration or evaporation.
c) Drying the VPO precursor and/or subjecting it to preliminary heat treatment, if appropriate partial preactivation by additional elimination of water from the VPO precursor. Mixing the dry VPO precursor powder with a divalent or trivalent iron compound present as a solid, before or after the step of drying and/or preliminary heat treatment, preferably after this step.
d) and e) as described above.

The catalytically active composition advantageously has an iron/vanadium atomic ratio of from 0.005 to <0.05, in particular from 0.01 to 0.035.

Furthermore, the addition of the divalent or trivalent iron compound can be effected by mixing with the previously calcined catalyst (process 4), i.e. the steps a) and b) are carried out in a manner analogous to process 3, the subsequent step c) is carried out in a manner analogous to process 1, followed by the step e) analogous to process 1, subsequently mixing-in of the Fe component and finally step d) analogous to process 1.

Furthermore, doping can also be effected via intercalation, as described, for example, in Satsuma et al., Catalysis Today 71 (2001) 161-167 (process 5).

Furthermore, the divalent or trivalent iron compound can also be introduced prior to the addition of the pentavalent phosphorus compound (process 6).

Furthermore, the addition of the divalent or trivalent iron compound can be carried out before the reduction of the V⁵⁺ component (process 7).

Particular preference is given to processes 1 to 3 according to the invention, in particular process 2.

Particular preference is given to a catalyst which can be produced by
a₁) reacting a pentavalent vanadium compound (e.g. V₂O₅) with an organic, reducing solvent (e.g. isobutanol) in the presence of a pentavalent phosphorus compound (e.g. orthophosphoric and/or pyrophosphoric acid) with heating to from 75 to 205° C., preferably to from 100 to 120° C.,
a₂) cooling the reaction mixture to advantageously from 40 to 900° C.,
a₃) adding iron(III) phosphate and
a₄) heating the reaction mixture to from 75 to 205° C., preferably to from 100 to 120° C.
b) isolating the vanadium-, phosphorus-, iron- and oxygen-comprising catalyst precursor
c) drying the VPO precursor and/or subjecting it to preliminary heat treatment, if appropriate partial preactivation by additional elimination of water from the VPO precursor
d) shaping the VPO precursor to give it a spherical, ring-shaped or shell-like structure for example
e) preactivating the shaped VPO precursor by heating in an atmosphere comprising oxygen, nitrogen, noble gases, carbon dioxide, carbon monoxide and/or water vapor, as described, for example, in WO03078310 on page 20, line 16 to page 21, line 35.

The invention further provides a process for preparing maleic anhydride by heterogeneously catalyzed gas-phase oxidation of a hydrocarbon having at least four carbon atoms by means of oxygen-comprising gases using the catalyst of the invention.

As reactors, use is generally made of shell-and-tube reactors. Suitable shell-and-tube reactors are described, for example, in EP-B 1 261 424 in paragraphs [0033] and [0034].

Hydrocarbons suitable for use in the process of the invention are aliphatic and aromatic, saturated and unsaturated hydrocarbons having at least four carbon atoms, for example those described in EP-B 1 261 424 in paragraph [0035].

The process for preparing maleic anhydride is known to those skilled in the art and is described, for example, in DE-A 10235355, EP-B 1 261 424 in paragraphs [0032] to and Ullmann's Encyclopedia of Industrial Chemistry, 6th Edition, 1999 electronic release, Chapter “Maleic- and Fumaric acids, Maleic anhydride—production”.

The process of the invention using the catalyst of the invention makes it possible to achieve a high yield of maleic anhydride at a high conversion. In particular, the use of iron(III) phosphate as doping component and use of the doping process 2 enables an improvement in yield to be achieved at comparable activity.

### EXAMPLES

### 1.1 Preparation of Catalyst Precursor 1 According to the Invention (by Process 1)

4602 kg of isobutanol were placed in an 8 m³ steel/enamel stirred vessel provided with baffles which could be heated externally by means of pressurized water and had been made inert by means of nitrogen. After starting up the three-stage impeller stirrer, the isobutanol was heated to 90° C. under reflux. At this temperature, the addition of 22.7 kg of Fe(III) phosphate (w_{Fe}=29.9% by weight) was then commenced. The addition of 690 kg of vanadium pentoxide via the feed screw was subsequently commenced. After about 213 of the desired amount of vanadium pentoxide had been added after about 20 minutes, the pumping-in of 805 kg of 105% strength phosphoric acid was commenced with further addition of vanadium pentoxide. After addition of the phosphoric acid, the reaction mixture was heated at about 100 to 108° C. under reflux and left under these conditions for 14 hours. The suspension was subsequently drained into a heated pressure filter which had been made inert by means of nitrogen and the solid was filtered off at a temperature of about 100° C. and a pressure above the pressure filter of up to 0.35 MPa abs. The filter cake was blown dry by continual introduction of nitrogen at 100° C. while stirring with a central stirrer whose height could be adjusted for about 1 hour. After blowing-dry, the filter cake was heated to about 155° C. and evacuated to a pressure of 15 kPa abs (150 mbar abs). Drying was carried out to a residual isobutanol content of <2% by weight in the dried catalyst precursor.

The Fe/V ratio was 0.016.

The dried powder was subsequently treated under air in a rotary tube having a length of 6.5 m, an internal diameter of 0.9 m and internal helices for 2 hours. The speed of rotation of the rotary tube was 0.4 rpm. The powder was fed into the rotary tube in an amount of 60 kg/h. Air was introduced at a rate of 100 m³/h. The temperature of the five equally long heating zones measured directly on the outside of the rotary tube were 250° C., 300° C., 345° C., 345° C. and 345° C.

### 1.2 Preparation of Catalyst Precursor 2 According to the Invention (by Process 2)

4602 kg of isobutanol were placed in an 8 m³ steel/enamel stirred vessel provided with baffles which could be heated externally by means of pressurized water and had been made inert by means of nitrogen. After starting up the three-stage impeller stirrer, the isobutanol was heated to 90° C. under reflux. At this temperature, the addition of 690 kg of vanadium pentoxide via the feed screw was then commenced. After about ⅔ of the desired amount of vanadium pentoxide had been added after about 20 minutes, the pumping-in of 805 kg of 105% strength phosphoric acid was commenced with furtheraddition of vanadium pentoxide. After addition of the phosphoric acid, the reaction mixture was heated at about 100 to 108° C. under reflux and left under these conditions for 14 hours. The hot suspension was then cooled to 60° C. over a period of 70-80 minutes and 22.7 kg of Fe(III) phosphate (w_{fe}=29.9% by weight) were added. After reheating to reflux over a period of 70 minutes, the suspension is boiled for a further one hour. The suspension was subsequently drained into a heated pressure filter which had been made inert by means of nitrogen and the solid was filtered off at a temperature of about 100° C. and a pressure above the pressure filter of up to 0.35 MPa abs. The filter cake was blown dry by continual introduction of nitrogen at 100° C. while stirring with a central stirrer whose height could be adjusted for about 1 hour. After blowing-dry, the filter cake was heated to about 155° C. and evacuated to a pressure of 15 kPa abs (150 mbar abs). Drying was carried out to a residual isobutanol content of <2% by weight in the dried catalyst precursor.

The Fe/V ratio was 0.016.

The dried powder was subsequently treated in air in a rotary tube having a length of 6.5 ml an internal diameter of 0.9 m and internal helices for 2 hours. The speed of rotation of the rotary tube was 0.4 rpm. The powder was fed into the rotary tube in an amount of 60 kg/h. Air was introduced at a rate of 100 m³/h. The temperature of the five equally long heating zones measured directly on the outside of the rotary tube were 2500° C., 3000° C., 3450° C., 3450° C. and 3450° C.

### 1.3 Preparation of Catalyst Precursor 3 According to the Invention (by Process 3)

4602 kg of isobutanol were placed in an 8 m³ steel/enamel stirred vessel provided with baffles which could be heated externally by means of pressurized water and had been made inert by means of nitrogen. After starting up the three-stage impeller stirrer, the isobutanol was heated to 90° C. under reflux. At this temperature, the addition of 690 kg of vanadium pentoxide via the feed screw was then commenced. After about 213 of the desired amount of vanadium pentoxide had been added after about 20 minutes, the pumping-in of 805 kg of 105% strength phosphoric acid was commenced with further addition of vanadium pentoxide. After addition of the phosphoric acid, the reaction mixture was heated at about 100 to 1080° C. under reflux and left under these conditions for 14 hours. The suspension was subsequently drained into a heated pressure filter which had been made inert by means of nitrogen and the solid was filtered off at a temperature of about 1000° C. and a pressure above the pressure filter of up to 0.35 MPa abs. The filter cake was blown dry by continual introduction of nitrogen at 1000° C. while stirring with a central stirrer whose height could be adjusted for about 1 hour. After blowing-dry, the filter cake was heated to about 155° C. and evacuated to a pressure of 15 kPa abs (150 mbar abs). Drying was carried out to a residual isobutanol content of <2% by weight in the dried catalyst precursor.

The dried powder was subsequently treated in air in a rotary tube having a length of 6.5 m, an internal diameter of 0.9 m and internal helices for 2 hours. The speed of rotation of the rotary tube was 0.4 rpm. The powder was fed into the rotary tube in an amount of 60 kg/h. Air was introduced at a rate of 100 m³/h. The temperature of the five equally long heating zones measured directly on the outside of the rotary tube were 250° C., 3000° C., 345° C., 3450° C. and 3450° C. The catalyst precursor was intimately mixed with Fe(III) phosphate (FePO₄x2H₂O) in an Fe/V atomic ratio of 0.016.

### 1.4 Preparation of Catalyst Precursor 4 According to the Invention (by Process 1)

A catalyst precursor was prepared by the method of example 1.1 using iron(III) acetylacetonate as iron starting material.

### 1.5 Preparation of Catalyst Precursor 5 According to the Invention (by Process 2)

A catalyst precursor was prepared by the method of example 1.2 using iron(III) acetylacetonate as iron starting material.

### 1.6 Preparation of Catalyst Precursor 6 According to the Invention (by Process 3)

A catalyst precursor was prepared by the method of example 1.3 using iron(III) acetylacetonate as iron starting material.

### 1.7 Preparation of Catalyst Precursor 7 According to the Invention

A catalyst precursor was prepared by the method of example 1.3 using iron(III) oxide as iron starting material.

### 1.8 Preparation of an Undoped Catalyst Precursor 8

An undoped catalyst precursor was prepared as described in EP-A 1485203 [page 38, paragraph 25 (example 8)].

### 2. Production of the Catalysts

### 2.1 Production of Catalysts 3a, 4a, 5a, 6a, 7a and 8a

400 g of the precursors 3 to 8 where intimately mixed with 10% by weight of graphite and processed further by means of a roller compactor (from Powtec) to produce a granulated material. For further processing, the sieve fraction 0.7-1.0 mm of the granulated material was used. 30 ml of the sieve fraction 0.7-1 mm of the granulated material were introduced into a vertical furnace (internal tube diameter: 26 mm, with a thermocouple sheath having a diameter of 4 mm). 25 standard l/h of air were passed over the precursor while the temperature was increased from room temperature to 250° C. (heating rater 5° C./min). After the temperature of 2500° C. had been reached, the furnace temperature was increased further to 330° C. at a heating rate of 2° C./min. This temperature was kept constant over a period of 40 minutes. While maintaining a volume flow of 25 standard l/h, the air was replaced by nitrogen/water (1:1) and the temperature was increased to 425° C. (heating rate. 30° C./min) and this temperature was maintained for a period of 180 minutes. The furnace was subsequently cooled to room temperature while continuing to pass 25 standard l/h N₂ over the catalyst.

### 2.2 Production of Catalysts 1a, 2a, 3b and 8b

The VPO precursor was intimately mixed with 1% by weight of graphite and compacted in a roller compactor. The fines having a particle size of <400 μm in the compacted material were sieved off and fed back into the compacting step. The coarse material having a particle size of >400 μm was mixed with a further 2% by weight of graphite and pelletized in a tableting machine to produce 5×3×2.5 mm hollow cylinders (external diameter×height×diameter of the central hole).

The 5×3×2.5 mm hollow cylinder obtained were calined as described in WO 03/78059, page 39 under example 9,

### 2.2.1 X-Ray Diffraction Analysis of Catalyst 2a

For the X-ray diffraction analysis, the catalyst 2a was pulverized and measured in a Siemens D5000 theta/theta X-ray powder diffractometer. The measurement parameters were as follows:

| Circle diameter | 435 | mm |
|---|---|---|
| X-radiation | CuKα | |
| Tube voltage | 40 | kV |
| Tube current | 40 | mA |
| Aperture | variable V2O | |
| Antiscatter aperture | variable V2O | |
| Secondary monochromator | graphite | |
| Monochromator aperture | 0.1 | mm |
| Scintillation counter | | |
| Detector apertures | 0.6 | mm |
| Step width | 0.02° | 2θ |
| Step mode | continuous | |
| Measurement time | 3.6 | s/step |
| Measurement speed | 0.33° | 2θ/min |

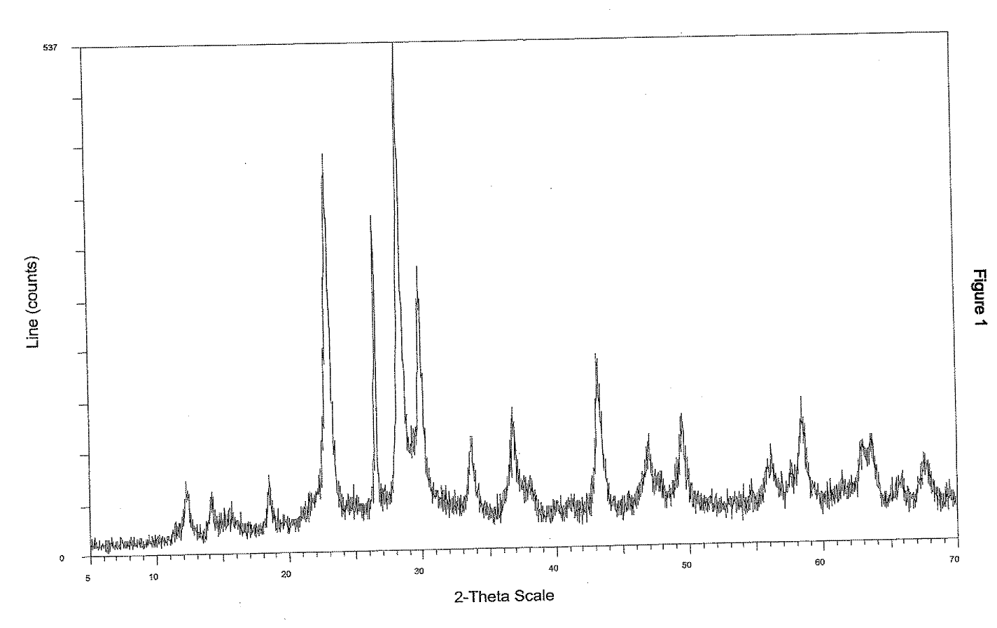

The XRD spectrum of catalyst 2a is shown in FIG. 1.

### 3. Process for Preparing Maleic Anhydride

### 3.1 Process for Preparing Maleic Anhydride with the Aid of an Experimental Screening Reactor Plant

The experimental plant was equipped with a feed metering unit and an electrically heated reactor tube. The reactor used had a tube length of 30 cm and an internal diameter of 11 mm. The temperature was measured externally on the heating shell of the reactor. 12 ml of catalyst in the form of granulated material having a particle size of 0.7-1.0 mm were in each case mixed with the same volume of inert material (steatite balls) and introduced into the reaction tube. The remaining empty volume was filled with inert material. The following reaction conditions were set: catalyst testing was carried out at a GHSV of 2000 h⁻¹, 2.0% by volume of n-butane, 3% by volume of water, 1.0 ppm by volume of triethyl phosphate and gauge pressure in the reactor of 1 bar. The performance of catalysts 3, 4, 5, 6, 7 and 8 was assessed after a period of operation of 75-150 hours at a conversion of about 85%. The results are shown in tables 1 and 3.

It may be noted that the screening experiments and the model tube experiment cannot be compared absolutely.

### 3.2 Process for Preparing Maleic Anhydride with the Aid of an Experimental Model Tube Plant

The experimental plant was equipped with a feed unit and a reactor tube. The plant was operated in a single pass, as described in EP-B 1 261 424.

The hydrocarbon was added in liquid form in a regulated amount by means of a pump. Air was added as oxygen-comprising gas in a regulated amount. Triethyl phosphate (TEP) was likewise added in a regulated amount, dissolved in water in liquid form. The shell-and-tube reactor unit comprised a shell-and-tube reactor having one reactor tube. The length of the reactor tube was 6.5 m, and the internal diameter was 22.3 mm. A multi thermocouple having 20 measurement points was located in a protective tube having an external diameter of 6 mm within the reactor tube. The reactor was heated by means of a heat transfer medium circuit having a length of 6.5 m. A salt melt was used as heat transfer medium.

The reaction gas mixture flowed through the reactor tube from the top downward. The upper 0.2 m of the 6.5 m long reactor tube remained unfilled. This was followed by a 0.3 m long preheating zone which was filled with shaped steatite bodies as inert material. The preheating zone was followed by the catalyst bed which comprised a total of 2180 ml of catalyst.

A gaseous product was taken off directly after the shell-and-tube reactor unit and passed to on-line analysis by gas chromatography. The main stream of the gaseous reactor output was discharged from the plant.

The measurements were carried out after a minimum period of operation of the catalysts 1, 2, 3 and 8 of 150 hours. The results are shown in table 2.

TABLE 1
Testing of a screening plant (2% by volume of n-butane, WHSV = 0.115
h⁻¹, 3% by volume of H₂O, 1 ppm by volume of TEP, pᵢₙ = 1 bar gauge)

| Precursor | 8 | 3 | 6 | 7 |
|---|---|---|---|---|
| Catalyst | 8a | 3a | 6a | 7a |
| according to the | no | yes | yes | yes |
| invention | | | | |
| BET surface | 26 | 24 | 25 | 23 |
| area [m²/g] | | | | |
| Conversion [%] | 85 | 85 | 85 | 85 |
| T_{Reactor} [° C.] | 419 | 405 | 408 | 401 |
| Y_{MA} [%] | 57.2 | 59.7 | 59.4 | 59.3 |
| S_{MA} [%] | 67.3 | 70.2 | 69.9 | 69.8 |

TABLE 2
Testing of a model tube plant (2% by volume of
n-butane, GHSV = 2000 h⁻¹, 3% by volume of H₂O,
2.25 ppm by volume of TEP, pᵢₙ = 2.3 bar gauge)

| Precursor | 8 | 1 | 2 | 3 |
|---|---|---|---|---|
| Catalyst | 8b | 1a | 2a | 3b |
| according to | no | yes | yes | yes |
| the invention | | | | |
| Doping method | | Process 1 | Process 2 | Process 3 |
| BET surface | 29 | 25 | 30 | 29 |
| area [m²/g] | | | | |
| Conversion [%] | 85 | 85 | 85 | 85 |
| T_{Reactor} [° C.] | 418 | 427 | 414 | 410 |
| Y_{MA} [%] | 54.9 | 55.8 | 57.1 | 55.4 |
| S_{MA} [%] | 64.6 | 65.6 | 67.2 | 65.2 |

TABLE 3
Testing screening plant (2% by volume of
n-butane, WHSV = 0.115 h⁻¹, 3% by volume of H₂O,
1 ppm by volume of TEP, pᵢₙ = 1 bar gauge)

| Precursor | 8 | 4 | 5 | 6 |
|---|---|---|---|---|
| Catalyst | 8a | 4a | 5a | 6a |
| according to | no | yes | yes | yes |
| the invention | | | | |
| Doping method | | Process 1 | Process 2 | Process 3 |
| BET surface | 26 | 25 | 23 | 26 |
| area [m²/g] | | | | |
| Conversion [%] | 85 | 85 | 85 | 85 |
| T_{Reactor} [° C.] | 419 | 406 | 405 | 408 |
| Y_{MA} [%] | 57.2 | 59.7 | 60.3 | 59.4 |
| S_{MA} [%] | 67.3 | 70.2 | 70.9 | 69.9 |

Definitions:
WHSV Mass of butane in g per g of catalyst per hour (weight hourly space velocity)
TEP Triethyl phosphate
pₑᵢₙ Reactor inlet pressure
GHSV Total gas flow in liters per liter of catalyst per hour (gas hourly space velocity)
T_{Reactor} [° C.] Temperature in the reactor
Y_{MA} [%] Yield of maleic anhydride
S_{MA} [%] Selectivity to maleic anhydride

1-12. (canceled)

13. A catalyst comprising a catalytically active composition, the catalytically active composition comprising vanadium, phosphorus, iron and oxygen, wherein the catalytically active composition has an iron:vanadium atomic ratio of 0.005 to <0.05, and wherein iron in the catalytically active composition is derived from an iron starting material comprising Fe(III) phosphate.

14. The catalyst according to claim 13, wherein the catalytically active composition has an iron:vanadium atomic ratio of 0.01 to 0.035.

15. The catalyst according to claim 13, wherein the catalytically active composition has a phosphorus:vanadium atomic ratio of 0.9 to 1.5, an average oxidation state of +3.9 to 4.4, a BET surface area of >15 m²/g, a pore volume of >0.1 m/g, and a bulk density of 0.5 to 1.5 kg/l.

16. A process comprising:
(a) reacting a pentavalent vanadium compound with an organic, reducing solvent in the presence of a pentavalent phosphorus compound with heating to a temperature of 75 to 205° C. to form a reaction mixture, cooling the reaction mixture to a temperature of 40 to 90° C., adding a divalent or trivalent iron compound to the cooled reaction mixture, and heating the reaction mixture to a temperature of 75 to 205° C. to form a vanadium-, phosphorus-, iron- and oxygen-comprising catalyst precursor;
(b) isolating the vanadium-, phosphorus-, iron- and oxygen-comprising catalyst precursor;
(c) subjecting the precursor to drying, a preliminary heat treatment, or both;
(d) shaping the precursor to provide precursor with a shape selected from the group consisting of spheres, rings, and shells; and
(e) preactivating the shaped catalyst precursor by heating in an atmosphere comprising one or more gases selected from the group consisting of oxygen, nitrogen, noble gases, carbon dioxide, carbon monoxide, water vapor, and mixtures thereof, to form a catalytically active composition having an iron:vanadium atomic ratio of 0.005 to 0.1.

17. The process according to claim 16, wherein the iron compound comprises one or more compounds selected from the group consisting of Fe(III) phosphate, Fe(III) acetylacetonate, Fe oxide, Fe vanadate, Fe molybdate, Fe(III) citrate, and Fe(II) oxalate.

18. The process according to claim 16, wherein the catalytically active composition has an iron:vanadium atomic ratio of 0.005 to <0.05.

19. The process according to claim 16, wherein the iron compound comprises Fe(III) phosphate.

20. A process comprising:
(a) reacting a pentavalent vanadium compound with an organic, reducing solvent in the presence of a pentavalent phosphorus compound with heating, to form a vanadium-, phosphorus-, and oxygen-comprising catalyst precursor;
(b) isolating the vanadium-, phosphorus- and oxygen-comprising catalyst precursor;
(c) subjecting the precursor to drying, a preliminary heat treatment, or both, and mixing the precursor with a divalent or trivalent iron compound;
(d) shaping the precursor to provide precursor with a shape selected from the group consisting of spheres, rings, and shells; and
(e) preactivating the shaped catalyst precursor by heating in an atmosphere comprising one or more gases selected from the group consisting of oxygen, nitrogen, noble gases, carbon dioxide, carbon monoxide, water vapor, and mixtures thereof, to form a catalytically active composition having an iron:vanadium atomic ratio of 0.005 to 0.1.

21. The process according to claim 20, wherein the iron compound comprises one or more compounds selected from the group consisting of Fe(III) phosphate, Fe(III) acetylacetonate, Fe oxide, Fe vanadate, Fe molybdate, Fe(III) citrate, and Fe(II) oxalate.

22. The process according to claim 21, wherein the catalytically active composition has an iron:vanadium atomic ratio of 0.005 to <0.05.

23. The process according to claim 21, wherein the iron compound comprises Fe(III) phosphate.

24. A process comprising:
(a) providing a hydrocarbon having at least four carbon atoms; and
(b) subjecting the hydrocarbon to gas-phase oxidation in the presence of a catalyst according to claim 13 to provide maleic anhydride.
